(19) United States
(12) Patent Application Publication
(10) Pub. No.: US 2023/0277320 A1
(43) Pub. Date: Sep. 7, 2023

(54) IMPLANT AND METHOD FOR MIDFACE REJUVENATION

(71) Applicant: Sea-Quan SU, Taipei (TW)

(72) Inventor: Yong-Kyu KIM, Seoul (KR)

(21) Appl. No.: 17/685,103

(22) Filed: Mar. 2, 2022

(51) Int. Cl. A61F 2/28; A61B 17/02

(52) U.S. Cl. CPC A61F 2/2875; A61B 17/0231; A61F 2002/2878

(57) ABSTRACT

An inferior orbital rim implant is provided. The inferior orbital rim implant is designed to rejuvenate the midface of a subject in need thereof. The implant allows a tear trough correction free of skin scars, with less operation time, and requires no stitches. A method for implanting the inferior orbital rim implant is also provided.

### TECHNICAL FIELD

The present disclosure relates to an implant, and more particularly to an inferior orbital rim implant for tear trough correction in midface rejuvenation of a subject in need thereof, and a method of tear trough correction in midface rejuvenation using the implant.

### DESCRIPTION OF RELATED ART

Eyelid surgeries, such as double fold surgery, upper blepharoplasty, lower blepharoplasty, tear trough surgery, etc. are some of the most popular aesthetic surgeries. In some scenarios, eyelid surgeries are focused on correcting tear trough caused by skin aging, bone aging, bony development, and/or position of an eyeball.

Three common approaches for correcting tear trough are provided. The first is subciliary incision lower blepharoplasty. This is the most popular surgical mean of the three, and usually comprises operations of skin excision, fat reposition or removal and canthopexy. This is an option for baggy lower eyelid but has several limitations; for example, the surgical technique is difficult to be performed and requires certain learning curve, and there are contraindications of the surgery such as decreased skin elasticity, exophthalmic eyeball and hypoplastic infraorbital rim. These contraindications can result in ectropion which is very difficult to be corrected.

The second is transconjunctival fat reposition. This approach is an option for patients who do not have excess skin and wish to avoid leaving scar on skin. However, the visual field during the surgery is small, making it difficult to operate and often resulting in under-correction and relapse.

The third is insertion of an implant to adepressed bony area (e.g., inferior orbital rim), which is often referred to as tear trough implant. It has several advantages except a foreign material is adopted. The surgical technique involved is easy, no scar is left on skin, and there is low possibility of ectropion. However, despite the plenty of tear trough implants designed and manufactured by different manufacturers, there remains a need for tear trough implants with an Asian fit or an East Asian fit, because there is a substantial difference between the aging processes of Asians and Caucasians.

Referring to FIG. 1A in the order of from left to right, it is illustrated that as an Asian patient ages, his/her inferior orbital rim is likely to retrude (also known as infra-orbital shortening or inferior rim absorption) and form a retruded part 10, thereby increasing laxity of his/her lower eyelid. In this case, zygoma of this Asian patient is relatively protruded, and anterior maxillary wall of this Asian patient is less absorbed or retracted that much.

On the other hand, as shown in the order of from left to right in FIG. 1B, it can be seen that as Caucasian patient ages, his/her anterior wall of maxilla is more likely to retract (also known as maxillary anterior wall absorption) and form a retracted part 10′, while the inferior orbital rim of the Caucasian patient is less likely to retrude. Therefore, the Caucasian patient is more likely to have increased laxity in cheeks, and jowl appears earlier as he/she ages.

Currently, most tear trough implants (also referred to as Caucasian tear trough implants herein) are designed to fit the aging Caucasian bony structure to rejuvenate the midface and to correct absorption or retraction of the maxillary anterior wall and less protruded zygoma. In this scenario, due to the large size of a Caucasian tear trough implant, it is usually implanted by making a subciliary incision in the lower eyelid of a Caucasian patient, and adjusting the subciliary incision to make a pocket large enough for fitting the Caucasian tear trough implant. The Caucasian tear trough implant is then inserted through the subciliary incision to cover and augment the maxillary anterior wall and part of zygoma (i.e., to fill up the retracted part 10′ shown in FIG. 1B), and is fixed by screws or stitches.

Therefore, if a surgeon would like to utilize the Caucasian tear trough implant for tear trough correction in an Asian patient, he or she will have to carve said Caucasian tear trough implant into desired shapes, while removing part of the anterior maxilla and zygoma from the Asian patient to enlarge the retruded part 10 shown in FIG. 1A, and then fit the carved Caucasian tear trough implant into said enlarged retruded part 10 (e.g., via the implanting steps of Caucasian tear trough implants as described above). Obviously, such approach for tear trough correction in an Asian patient is not only time consuming, but also complicated that includes unnecessary and excessive steps, and may leave skin scar on patients.

It is thus still an unmet need to design an inferior orbital rim implant for Asian patients for use in tear trough correction.

### SUMMARY

An inferior orbital rim implant for tear trough correction of a human midface is provided, which comprises: a bottom surface including: a long edge; a first curved edge and a second curved edge opposed to the first curved edge, wherein a first end of each of the first curved edge and the second curved edge meets either of two ends of the long edge, respectively; a short edge opposed to and in parallel with the long edge, wherein a second end of each of the first curved edge and the second curved edge meets either of two ends of the short edge, respectively; a flat side surface intersected substantially perpendicularly with the bottom surface along the long edge; a curved side surface opposed to the flat side surface and intersected substantially perpendicularly with the bottom surface along the first curved edge, the short edge and the second curved edge; and a cambered top surface opposed to the bottom surface and intersected substantially perpendicularly with the flat side surface and the curved side surface, respectively.

A method for implanting an inferior orbital rim implant for tear trough correction of a human midface is also provided, which comprises: providing the inferior orbital rim implant as described above; determining a pocket size for implantation of the inferior orbital rim implant; locating a lower border of a tarsal plate; making an incision close to the lower border of the tarsal plate; performing a dissection to create a pocket having the determined pocket size to a supraperiosteal plane of an inferior orbital rim of a human midface; inserting the inferior orbital rim implant through the incision into the pocket; and fixing the inferior orbital rim implant to a periosteum.

In some embodiments, the dissection is made between orbicularis oculi muscle and orbital septum to arcus marginalis. In some embodiments, the dissection plane is then changed over to periosteum. In some embodiments, the pocket is about 2 to 3 mm larger than the implant size, such that the pocket is in a size to receive the implant without excess space and to prevent movement of the implant to an adjacent area.

The present disclosure therefore provides an implant and a method for tear trough correction that is achieved by a transconjunctival approach (e.g., preseptal inferior fornix transconjunctival surgery). The tear trough correction carried out by the present disclosure is free of skin scars, has less operation time, and requires no stitches.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings, wherein:

FIGS. 1A and 1B are schematic diagrams illustrating aging difference of bony structures of faces of Asian and Caucasian patients, respectively;

FIGS. 2A to 2D are schematic diagrams illustrating an inferior orbital rim implant in accordance with embodiments of the present disclosure;

FIG. 3 is a flow chart describing steps to implant an inferior orbital rim implant in accordance with embodiments of the present disclosure;

FIGS. 4A to 4D are schematic diagrams illustrating steps for implanting an inferior orbital rim implant in accordance with embodiments of the present disclosure; and

FIGS. 5A and 5B are schematic diagrams illustrating the effect before and after an Asian patient undergo tear trough correction of an Asian patient with an inferior orbital rim implant in accordance with embodiments of the present disclosure.

### DETAILED DESCRIPTION OF THE EMBODIMENTS

The present disclosure is described by the following embodiments. Those with ordinary skill in the art can readily understand other advantages and functions of the present disclosure after reading the disclosure of this specification. The present disclosure may also be practiced or applied with other different implementations.

It should be appreciated that the structures, proportions, size and the like of the figures in the present application are intended to be used in conjunction with the disclosure of the specification. They are not intended to limit the disclosure and therefore do not represent any substantial technical meanings. Numerous modifications and variations can be devised without departing from the scope of the present disclosure. Further, the terms such as “first,” “second,” “one,” “a” etc. are used to distinguish one element from another and should not be construed to limit the scope of the present disclosure.

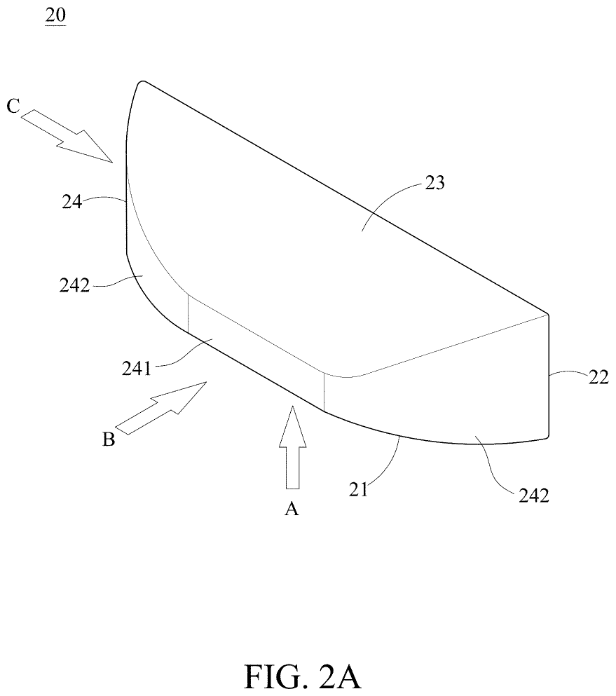

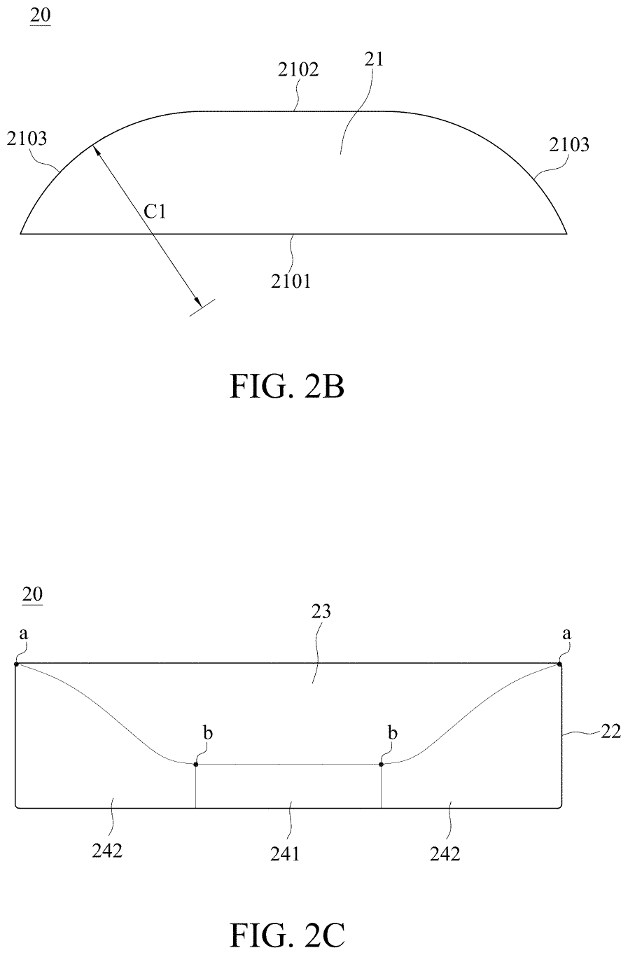

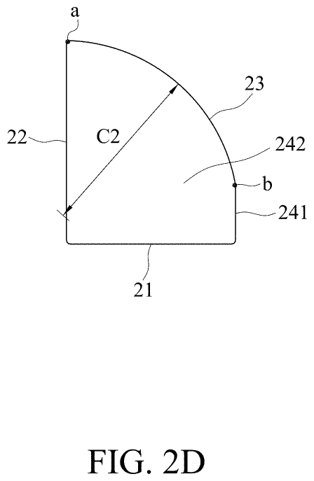

Referring to FIGS. 2A to 2D, in which an inferior orbital rim implant 20 suitable for tear trough correction is illustrated. Specifically, the inferior orbital rim implant 20 is a single piece material composed of silicone, Dacron, synthetic biogenerated graft, porcine graft, human graft, Gortex, expanded polytetrafluoroethylene, or other suitable materials that exhibit a low risk of rejection reaction from a human body.

In at least one embodiment of this disclosure, the inferior orbital rim implant 20 has a bottom surface 21, a flat side surface 22 intersected perpendicularly with the bottom surface 21, a curved side surface 24 opposed to the flat side surface 22 and intersected perpendicularly with the bottom surface 21, and a cambered top surface 23 opposed to the bottom surface and intersected perpendicularly with the flat side surface 22 and the curved side surface 24 respectively.

In at least one embodiment of this disclosure, the bottom surface 21 of the inferior orbital rim implant 20 (observed from direction A shown in FIG. 2A and further detailed in FIG. 2B) has a shape of an obround being halved along its long axis. For example, the bottom surface 21 has a long edge 2101 extending along the long axis of an obround shape that defines the overall width of the inferior orbital rim implant 20, two curved edges 2103 opposing to each other and having a first end that meets with either of the two ends of the long edge 2101 respectively, and a short edge 2102 opposing to and in parallel with the long edge 2101 which closes up the shape of the bottom surface 21 by having both its ends meet with either of the second end of the two curved edges 2103, respectively. For the convenience of future references, the curved edges 2103 will exhibit a curvature corresponding to a central angle radius cited as C1, where the configuration of the central angle radius C1 will be further discussed in later part of this disclosure.

Further, the curved side surface 24 of the inferior orbital rim implant 20 (observed from direction B shown in FIG. 2A and further detailed in FIG. 2C) comprises three subsurfaces, namely, flat subsurface 241 and two cambered subsurfaces 242. For example, referring to FIGS. 2A to 2C, the flat subsurface 241 has a quadrilateral shape that intersected perpendicularly with the bottom surface 21 along the short edge 2102, and thus exhibits the same length as the short edge 2102. On the other hand, the two cambered subsurfaces 242 have shapes of annular sector that intersected perpendicularly with the bottom surface 21 and cambered along the two curved edges 2103, respectively. The two cambered subsurfaces 242 thus exhibit a surface curvature corresponding to the curvature of the curved edges 2103. It should also be noted that as shown in FIG. 2C, the height of the two cambered subsurfaces 242 gradually decreased from the side that meets with the flat side surface 22 (which defines the overall height of the inferior orbital rim implant 20) to the side that meets with the flat subsurface 241.

Moreover, the cambered top surface 23 of the inferior orbital rim implant 20 is shaped as a cambered annular sector that corresponds to the shape of the bottom surface 21 (observed from direction A shown in FIG. 2A). In detail, the cambered top surface 23 (observed from directions B and C shown in FIG. 2A and further detailed in FIGS. 2C and 2D) has a surface curvature cambered from the height (measured from the bottom surface 21) of the flat side surface 22 (i.e., the tallest point “a,” also defined as the overall height of the inferior orbital rim implant 20) to the height of the flat subsurface 241 (i.e., the lowest point “b”). When observed from direction C of the inferior orbital rim implant 20 (as shown in FIG. 2D), the cambered top surface 23 exhibits a surface curvature corresponding to a central angle radius cited as C2, which is approximately the overall thickness of the inferior orbital rim implant 20 (i.e., the distance measured from the short edge 2102 to the long edge 2101 of the bottom surface 21). Similarly, the configuration of the central angle radius C2 will also be further discussed in later part of this disclosure. It should also be noted that the highest edge (i.e., the edge at the highest point “a”) of the cambered top surface 23 shares the same length as the flat side surface 22, i.e., the length of the long edge 2101 of the bottom surface 21 shown in FIG. 2B, and the lowest edge (i.e., the edge at the lowest point “b”) of the cambered top surface 23 shares the same length as the flat subsurface 241 of the curved side surface 24, i.e., the length of the short edge 2102 of the bottom surface 21 shown in FIG. 2B. As further shown in FIGS. 2C and 2D, the side edges (i.e., the two edges from the highest point “a” to the lowest point “b”) of the cambered top surfaces 23 exhibit a lateral curvature (viewed from direction B shown in FIG. 2A) corresponding to the central angle radius C1 and a horizontal curvature (viewed from direction C shown in FIG. 2A) corresponding to the central angle radius C2.

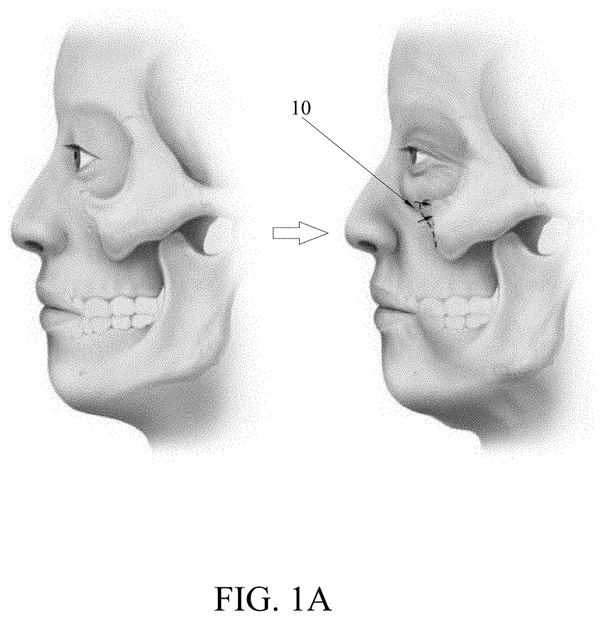

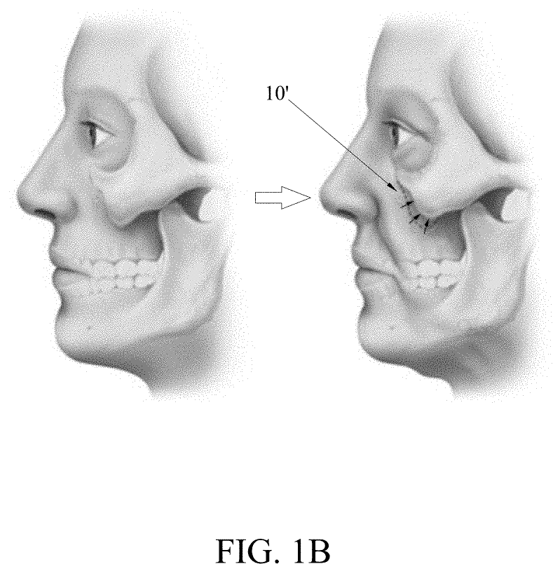

In order for the inferior orbital rim implant 20 to fit the retruded part 10 of an inferior orbital rim in a patient as shown in FIG. 1A at different aging stages and with different sizes of eyes and rims, the inferior orbital rim implant 20 of this disclosure may have various default dimensions. The dimensions of the implant of the present disclosure are designated so as to allow a transconjunctival approach for implantation. For example, the overall height of the inferior orbital rim implant 20 is designed based on the location of the infraorbital foramen of the maxilla, and the overall thickness of the inferior orbital rim implant 20 is designed based on the length of the orbital floor. Also, the overall width of the inferior orbital rim implant 20 is designed based on the length of the orbital rim exclusive of the zygoma. In at least one embodiment of this disclosure, based on the average size of the retruded part 10 of the inferior orbital rim of an Asian patient in early stages of aging (e.g., a person in his/her 20s or 30s), the inferior orbital rim implant 20 may be designed to have the following dimensions.

With regard to the bottom surface 21, the length of the long edge 2101 is about 20 millimeters (mm) with a tolerance range from plus 0.10 mm to minus 0.50 mm (i.e., the overall width of the inferior orbital rim implant 20); the length of the short edge 2102 is about 5 mm; the central angle radius C1 of the curved edges 2103 is about R9.03 with an arc length (when projected onto the long edge 2101) being 7.5 mm; and the distance measured from the long edge 2101 to the short edge 2102 of the bottom surface 21 (i.e., the overall thickness of the inferior orbital rim implant 20) is 4 mm with a tolerance range from plus to minus 0.10 mm.

With regard to the flat side surface 22, the height (measured from the bottom surface 21) of the flat side surface 22 is about 5 mm with a tolerance range from plus to minus 0.10 mm (i.e., the overall height of the inferior orbital rim implant 20); and the length of the flat side surface 22 is the same as that of the long edge 2101 of the bottom surface 21.

With regard to the curved side surface 24, the height (measured from the bottom surface 21) of the flat subsurface 241 is about 1.8 mm; the height of the cambered subsurfaces 242 is configured to gradually increase from about 1.8 mm (where they meet with the flat subsurface 241) to about 5 mm (where they meet with the flat side surface 22), respectively; and the surface curvature of the cambered subsurfaces 242 should correspond to the central angle radius C1 of the curved edges 2103 of the bottom surface 21, which is about R9.03 in this case.

With regard to the cambered top surface 23, it exhibits a surface curvature that corresponds to the central angle radius C2 of about R4.1, which is approximately the overall thickness of the inferior orbital rim implant 20; the highest edge of the cambered top surface 23 has the same length as that of the flat side surface 22; the lowest edge of the cambered top surface 23 has the same length as that of the flat subsurface 241 of the curved side surface 24; and the side edges of the cambered top surface 23 exhibit a lateral curvature corresponding to the central angle radius C1 (e.g., R9.03) and a horizontal curvature corresponding to the central angle radius C2 (e.g., R4.1).

In some examples, based on the average size of the retruded part 10 of the inferior orbital rim of an Asian patient in later stages of aging (e.g., a person in his/her 40s or 50s), the inferior orbital rim implant 20 may be designed to have the following dimensions.

With regard to the bottom surface 21, the length of the long edge 2101 is about 22.5 millimeters (mm) with a tolerance range from plus 0.10 mm to minus 0.30 mm; the length of the short edge 2102 is about 7.5 mm; the central angle radius C1 of the curved edges 2103 is about R8.13 with an arc length (when projected onto the long edge 2101) being about 7.5 mm; and the distance measured from the long edge 2101 to the short edge 2102 of the bottom surface 21 (i.e., the overall thickness of the inferior orbital rim implant 20) is about 5 mm with a tolerance range from plus to minus 0.10 mm.

With regard to the flat side surface 22, the height (measured from the bottom surface 21) of the flat side surface 22 is about 6 mm with a tolerance range from plus to minus 0.10 mm (i.e., the overall height of the inferior rim implant 20); and the length of the flat side surface 22 is the same as that of the long edge 2101 of the bottom surface 21.

With regard to the curved side surface 24, the height (measured from the bottom surface 21) of the flat subsurface 241 is about 1.85 mm; the height of the cambered subsurfaces 242 is configured to gradually increase from about 1.85 mm (where they meet with the flat subsurface 241) to about 6 mm (where they meet with the flat side surface 22), respectively; and the surface curvature of the cambered subsurfaces 242 should correspond to the central angle radius C1 of the curved edges 2103 of the bottom surface 21, which is about R8.13 in this case.

With regard to the cambered top surface 23, it exhibits a surface curvature that corresponds to the central angle radius C2 of about R5.09, which is approximately the overall thickness of the inferior orbital rim implant 20; the highest edge of the cambered top surface 23 has the same length as that of the flat side surface 22; the lowest edge of the cambered top surface 23 has the same length as that of the flat subsurface 241 of the curved side surface 24; and the side edges of the cambered top surface 23 exhibit a lateral curvature corresponding to the central angle radius C1 (e.g., R8.13) and a horizontal curvature corresponding to the central angle radius C2 (e.g., R5.09).

In some examples, based on the average size of the retruded part 10 of the inferior orbital rim of an Asian patient in late stages of aging (e.g., a person in his/her 60s or above), the inferior orbital rim implant 20 may be designed to have the following dimensions.

With regard to the bottom surface 21, the length of the long edge 2101 is about 25 millimeters (mm) with a tolerance range from plus 0.10 mm to minus 0.20 mm; the length of the short edge 2102 is about 10 mm; the central angle radius C1 of the curved edges 2103 is about R7.69 with an arc length (when projected onto the long edge 2101) being about 7.5 mm; and the distance measured from the long edge 2101 to the short edge 2102 of the bottom surface 21 (i.e., the overall thickness of the inferior orbital rim implant 20) is about 6 mm with a tolerance range from plus to minus 0.10 mm.

With regard to the flat side surface 22, the height (measured from the bottom surface 21) of the flat side surface 22 is about 7 mm with a tolerance range from plus to minus 0.10 mm (i.e., the overall height of the inferior rim implant 20); and the length of the flat side surface 22 is the same as that of the long edge 2101 of the bottom surface 21.

With regard to the curved side surface 24, the height (measured from the bottom surface 21) of the flat subsurface 241 is about 1.9 mm; the height of the cambered subsurfaces 242 is configured to gradually increase from about 1.9 mm (where they meet with the flat subsurface 241) to about 7 mm (where they meet with the flat side surface 22), respectively; and the surface curvature of the cambered subsurfaces 242 should correspond to the central angle radius C1 of the curved edges 2103 of the bottom surface 21, which is about R7.69 in this case.

With regard to the cambered top surface 23, it exhibits a surface curvature that corresponds to the central angle radius C2 of about R6.08, which is approximately the overall thickness of the inferior orbital rim implant 20; the highest edge of the cambered top surface 23 has the same length as that of the flat side surface 22; the lowest edge of the cambered top surface 23 has the same length as that of the flat subsurface 241 of the curved side surface 24; and the side edges of the cambered top surface 23 exhibit a lateral curvature corresponding to the central angle radius C1 (e.g., R7.69) and a horizontal curvature corresponding to the central angle radius C2 (e.g., R6.08).

It should be appreciated that the aforementioned dimensions of the inferior orbital rim implant 20 are only for illustrative purposes, and they are not meant to restrict the scope of the present disclosure. In other words, the various default dimensions of the inferior orbital rim implant 20 may be designed differently in advance by the manufacture or surgeon on demands, as long as the inferior orbital rim implant 20 meets the requirement of an Asian patient in need of tear trough correction.

As mentioned, the inferior orbital rim implant 20 of this disclosure is suitable to be implanted via a transconjunctival approach (e.g., a preseptal inferior fornix transconjunctival surgery), which is a safer procedure than subciliary incision for lower eyelid as mentioned above that is less likely to injure skin, muscle and septum of the anterior and middle labella of lower eyelid.

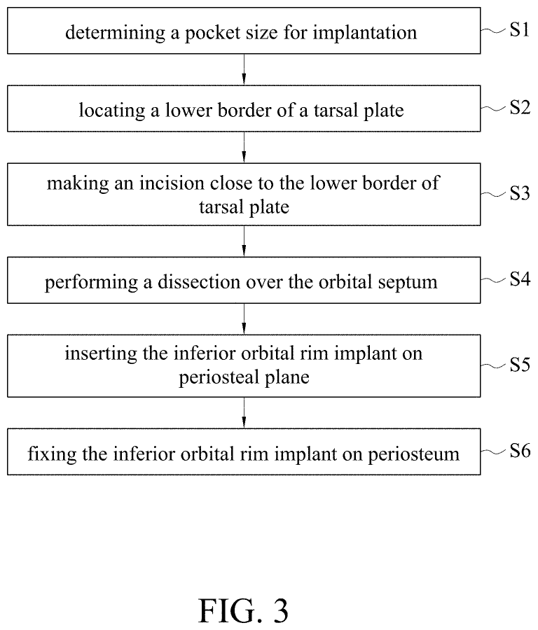

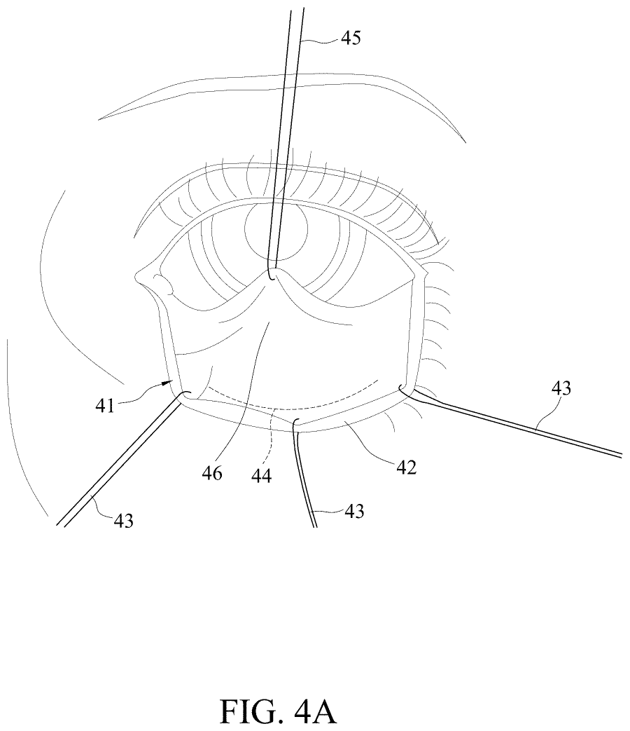

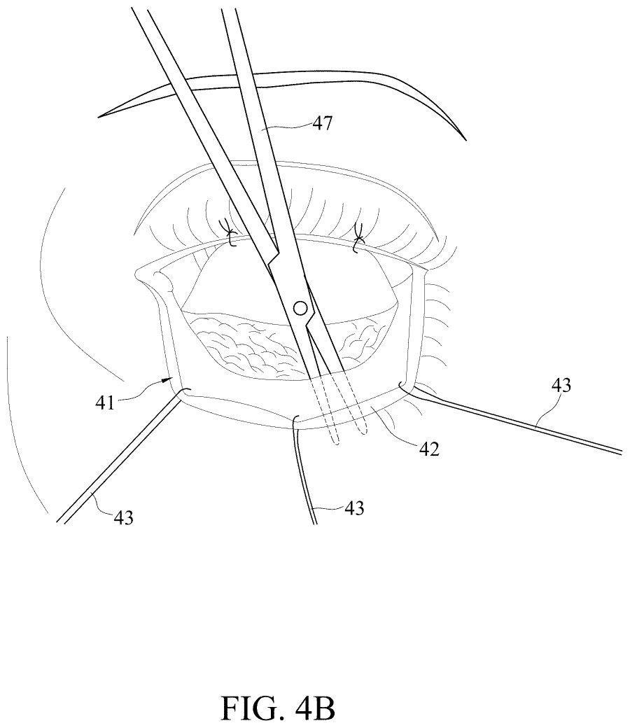

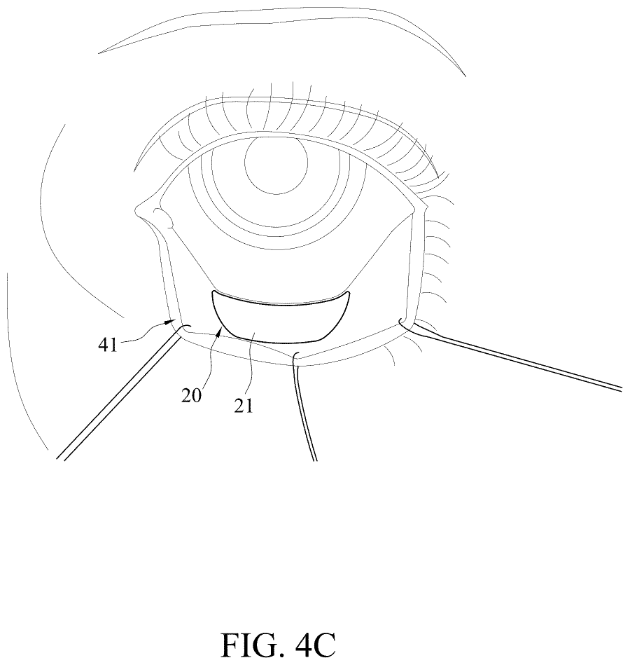

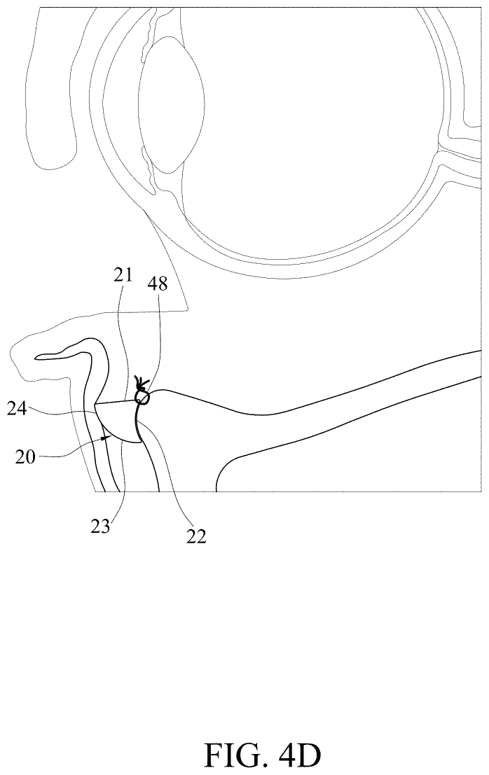

FIG. 3 is a flow chart describing exemplary steps for implanting the inferior orbital rim implant 20 through a transconjunctival approach. Said steps may be understood by reference to FIGS. 4A to 4D below. In an embodiment, said transconjunctival approach is mainly based on preseptal inferior fornix transconjunctival surgery. However, similar transconjunctival approaches for implanting the inferior orbital rim implant 20 may also be utilized, and the description below is only meant for illustrative purposes, not for restricting the scope of this disclosure.

In step S1, a pocket size for implanting the inferior orbital rim implant 20 is determined. Specifically, the pocket size should be able to accommodate design of said inferior orbital rim implant 20 (e.g., the different dimensions of the inferior orbital rim implant 20 as discussed above).

In step S2 (with reference to FIG. 4A), a lower border of the tarsal plate 42 of the lower eyelid 41 is located, so as to decide on an incision position. As illustrated in FIG. 4A, when the eyelid margin of the lower eyelid 41 of a patient is everted by having a plurality of traction sutures 43 placed through the lower eyelid 41, and the posterior edge of the conjunctiva 46 tented and secured by a stay traction suture 45, the lower border of the tarsal plate 42 may be identified.

In step S3 (continuing with reference to FIG. 4A), an incision 44 is made close to the lower border of the tarsal plate 42. In some embodiments, the incision 44 is made 3 to 4 mm below the eyelid margin of the lower eyelid 41 using laser or electrocautery.

In step S4 (with reference to FIG. 4B), dissection is performed over an orbital septum along the incision 44 to create a pocket having the pocket size for inserting the inferior orbital rim implant 20 (as discussed in step S1). As illustrated in FIG. 4B, with the lower eyelid 41 staying retracted by instruments (e.g., the traction sutures 43 and stay traction suture 45 described above or the like), a dissection (e.g., by using scissors 47) may be performed between the orbital septum and orbicularis oculi muscle. For example, the dissection is made at a position where no blood vessel is present. In some embodiments, the dissection plane will reach to arcus marginalis and then to a supraperiosteal plane, and the dissection will continue to perform caudally and horizontally for about 5 to 6 mm to make the pocket for implanting the inferior orbital rim implant 20.

In step S5 (with reference to FIGS. 4C and 4D), an inferior orbital rim implant 20 is inserted onto the periosteal plane. As shown in FIG. 4C, the inferior orbital rim implant 20 is inserted in the direction where the bottom surface 21 is facing upwards (with respect to the cranio-caudal axis of the patient in a standing position), meaning that (referring to the lateral view of insertion of the inferior orbital rim implant 20 as illustrated in FIG. 4D) the curved side surface 24 will abut muscle (e.g., orbicularis oculi muscle), and the flat side surface 22 will abut bone (e.g., periosteal plane). Also, the cambered top surface 23 will abut the deepest region of the pocket at the inferior orbital rim of the patient.

In step S6, implantation of the inferior orbital rim implant 20 is completed by fixing the inferior orbital rim implant 20 to the periosteum of the inferior orbital rim with a suture 48. For example, the implant is fixed to periosteum with a 6-0 non-absorbable nylon suture at 2 or 3 points to prevent movement immediately after surgery. To this stage, there is no need to stitch or repair the incision 44, and the incision 44 will be closed and healed spontaneously after implantation. In some embodiments, taping on skin is helpful for preventing edema and stabilizing the implant position for 1 day.

Through steps S1 to S6 described above, the implantation of the inferior orbital rim implant 20 may be inserted through small pockets, where less tissue injury will occur, and no skin scar will be made or visible. Further, the transconjunctival approach as described above only requires quick operation time with no stitches.

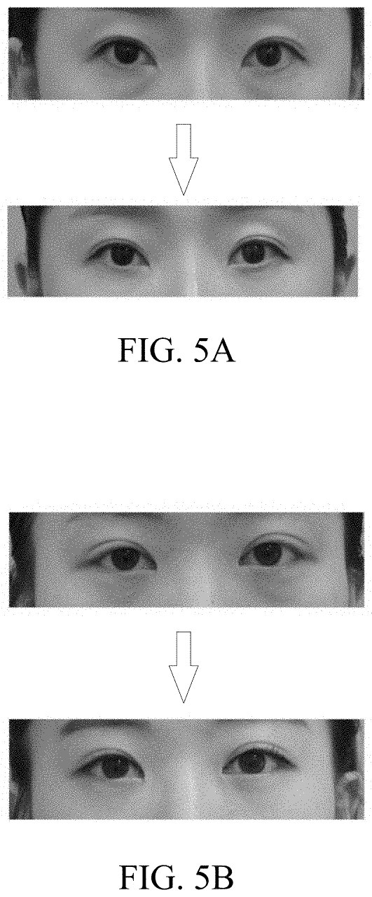

FIGS. 5A and 5B illustrate a comparison of midface of two patients before (left photograph) and after the implantation (right photograph) of the inferior orbital rim implant 20. From here, it is clear that the midface of both the patients looks more rejuvenated after the implantation, where no skin scar is visible after the transconjunctival approach.

In at least one embodiment of the present disclosure, the inferior orbital rim implant and method for implanting the same are designed to fit aging Asian bony structure to rejuvenate the midface of an Asian patient. Since the inferior orbital rim implant is smaller than a Caucasian tear trough implant, it requires only transconjunctival approaches that are free of skin scars, takes less operation time, and requires no stitches.

The foregoing descriptions of the embodiments are illustrated to disclose the features and functions of the present disclosure and not restrictive of the scope of the present disclosure. It should be understood to those skilled in the art that all modifications and variations according to the present disclosure should fall within the scope of the appended claims.

What is claimed is:

1. An inferior orbital rim implant for tear trough correction in a human midface, comprising:
a bottom surface including:
a long edge;
a first curved edge and a second curved edge opposed to the first curved edge, wherein a first end of each of the first curved edge and the second curved edge meets either of two ends of the long edge, respectively; and
a short edge opposed to and in parallel with the long edge, wherein a second end of each of the first curved edge and the second curved edge meets either of two ends of the short edge, respectively;
a flat side surface intersected substantially perpendicularly with the bottom surface along the long edge;
a curved side surface opposed to the flat side surface and intersected substantially perpendicularly with the bottom surface along the first curved edge, the short edge and the second curved edge; and
a cambered top surface opposed to the bottom surface and intersected substantially perpendicular with the flat side surface and the curved side surface, respectively.

2. The inferior orbital rim implant of claim 1, wherein a length of the long edge defines an overall width of the inferior orbital rim implant, wherein a height of the flat side surface defines an overall height of the inferior orbital rim implant, and wherein a distance between the long edge and the short edge defines an overall thickness of the inferior orbital rim implant.

3. The inferior orbital rim implant of claim 2, wherein the curved side surface comprises:
a flat subsurface intersected substantially perpendicularly with the bottom surface along the short edge, wherein a height of the flat subsurface is lower than the overall height;
a first cambered subsurface intersected substantially perpendicularly with the bottom surface along the first curved edge; and
a second cambered subsurface intersected substantially perpendicularly with the bottom surface along the second curved edge.

4. The inferior orbital rim implant of claim 3, wherein the first cambered subsurface and the second cambered subsurface are of a shape of a cambered annular sector gradually increasing in height from the height of the flat subsurface to the overall height of the inferior orbital rim implant.

5. The inferior orbital rim implant of claim 3, wherein at least one of the first cambered subsurface and the second cambered subsurface exhibit a surface curvature corresponding to a first central angle radius.

6. The inferior orbital rim implant of claim 3, wherein the cambered top surface exhibits a surface curvature cambered from the overall height to the height of the flat subsurface, and wherein the surface curvature of the cambered top surface corresponds to a second central angle radius corresponding to the overall thickness.

7. The inferior orbital rim implant of claim 6, wherein the cambered top surface comprises:
a highest edge having the length of the long edge of the bottom surface;
a lowest edge having the length of the short edge of the bottom surface; and
a first side edge and a second side edge opposed to the first side edge, wherein the first side edge and the second side edge exhibit a lateral curvature corresponding to curvatures of the first curved edge and the second curved edge of the bottom surface, respectively, and wherein the first side edge and the second side edge exhibit a horizontal curvature corresponding to the second central angle radius.

8. The inferior orbital rim implant of claim 2, wherein the overall height corresponds to a location of infraorbital foramen of maxilla of the human midface, wherein the overall thickness corresponds to a length of an orbital floor of the human midface, and wherein the overall width corresponds to a length of an orbital rim of the human midface exclusive of zygoma.

9. The inferior orbital rim implant of claim 1, which is made of a single piece material composed by one of silicone, Dacron, synthetic biogenerated graft, porcine graft, human graft, Gortex, expanded polytetrafluoroethylene, or any combination thereof.

10. A method for tear trough correction of a human midface, comprising:
providing the inferior orbital rim implant of claim 1;
determining a pocket size for implantation of the inferior orbital rim implant;
locating a lower border of a tarsal plate on the human midface;
making an incision close to the lower border of the tarsal plate;
performing a dissection along the incision to create a pocket having the pocket size to a supraperiosteal plane of an inferior orbital rim of the human midface;
inserting the inferior orbital rim implant through the incision into the pocket; and
fixing the inferior orbital rim implant to a periosteum.

11. The method of claim 10, wherein locating the lower border of the tarsal plate on the human midface further comprises:
everting an eyelid margin of a lower eyelid of the human midface and securing the lid margin via traction sutures placed through the lower eyelid;
having a posterior edge of a conjunctiva of the human midface tented and secured by a stay traction suture, such that the conjunctiva is lifted; and
identifying a position of the lower border of the tarsal plate.

12. The method of claim 10, wherein making the incision close to the lower border of the tarsal plate is performed by laser or electrocautery, and wherein the incision is 3 to 4 mm below an eyelid margin of a lower eyelid of the human midface.

13. The method of claim 10, wherein performing the dissection along the incision to create the pocket having the pocket size to the supraperiosteal plane of the inferior orbital rim of the human midface further comprises:
performing the dissection between an orbital septum and an orbicularis oculi muscle of the inferior orbital rim;
reaching a dissection plane to arcus marginalis of the inferior orbital rim;
changing the dissection plane to the supraperiosteal plane of the inferior orbital rim; and
performing the dissection caudally and horizontally for 5 to 6 mm to make the pocket.

14. The method of claim 10, wherein implanting the inferior orbital rim implant through the incision into the pocket further comprises having a curved side surface of the inferior orbital rim implant abut an orbicularis oculi muscle of the inferior orbital rim within the pocket, and having a flat side surface of the inferior orbital rim implant abut the supraperiosteal plane of the inferior orbital rim within the pocket.
